(19) United States
(12) Patent Application Publication
(10) Pub. No.: US 2023/0236172 A1
(43) Pub. Date: Jul. 27, 2023

(54) T Cells That Respond To Patient Neoepitopes

(71) Applicant: NantCell, Inc., Culver City, CA (US)

(72) Inventor: Peter Sieling, Culver City, CA (US)

(73) Assignee: NantCell, Inc., Culver City, CA (US)

(21) Appl. No.: 17/906,782

(22) PCT Filed: Mar. 4, 2021
(86) PCT No.: PCT/IB21/51786

§ 371 Date: Sep. 20, 2022

Related U.S. Application Data
(60) Provisional application No. 62/992,794, filed on Mar. 20, 2020.
(60) Provisional application No. 63/003,496, filed on Apr. 1, 2020.

(51) Int. Cl. G01N 33/50

(52) U.S. Cl. CPC G01N 33/5091; G01N 2800/24; G01N 2800/50

(57) ABSTRACT

Compositions and methods are presented that allow for detection and prediction of an immune response in a subject that is selected to receive or that has received a vaccine. In selected embodiments, whole blood is used as starting material to obtain both dendritic cells and T cells, and synthetic or recombinant polypeptide(s) are used that include an antigen of the vaccine. The dendritic cells are then exposed to the synthetic or recombinant polypeptide(s), and thusly exposed dendritic cells are combined with the T cells to generate antigen reactive T cells. For detection or quantification, the antigen reactive T cells are expanded in vitro prior to ELISPOT or FACS analysis. Advantageously, such systems and methods are especially suitable for ascertaining an immune response against cancer antigens following vaccination with an anti-cancer vaccine.

This application claims priority to our co-pending U.S. provisional patent application Ser. No. 62/992,794, filed Mar. 20, 2020, and 63/003,496, filed Apr. 1, 2020. Both of these applications are incorporated by reference herein in its entirety, including the drawings.

### SEQUENCE LISTING

The content of the ASCII text file of the sequence listing named Seq_listing_ST25, which is 17 kb in size was created on 02/26/2020 and electronically submitted via EFS-Web along with the present application is incorporated by reference in its entirety

### FIELD OF THE INVENTION

The present disclosure relates to compositions and methods of ascertaining or predicting an immune response against an antigen, and most typically a therapeutic antigen in an antitumor vaccine.

### BACKGROUND OF THE INVENTION

The background description includes information that may be useful in understanding the present disclosure. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

While numerous cancer vaccines have been developed targeting a number of molecular targets, clinical success is often unpredictable and may vary significantly from one antigen to another or among different patients where common antigens are targeted. Indeed, predicting the likelihood of a therapeutic effect (whether or not a functional immune response against the tumor is elicited) has been elusive, and therapeutic effect, and with that a determination as to continued treatment with the vaccine, is in many cases only based on quantitation of retarded or reversed tumor growth. Consequently, significant time will have lapsed between start of treatment and a first significant indication of treatment effect.

More recently, T cells from the tumor microenvironment (e.g., tumor infiltrating lymphocytes (TIL)) or adjacent lymphatic organs have been examined to determine reactivity and quantity of antigen reactive T cells. However, collecting these cells can be highly invasive and uncomfortable for the patient, and most typically such cells are obtained from tumor biopsies or otherwise surgically removed tumor sample. For example, antigen reactive tumor infiltrating lymphocytes have been used as therapeutic entities in combination with further immune modulatory agents as reported in Nature Medicine (URL: doi.org/10.1038/s41591-018-0040-8). While notably effective, the TIL were obtained from a surgical sample.

Assessment of an immune response in vitro has been described in U.S. Pat. No. 8,697,371 where an artificial immune system was proposed comprising T cells, B cells, and dendritic cells for in vitro testing of vaccines, adjuvants, immunotherapy candidates, biologics, etc. Unfortunately, such system will not provide an indication of a likely immune response in vivo where a subject was already exposed to an antigen or anti-cancer vaccine. Likewise, due to high inter-subject variability results from such in vitro tests will not necessarily equally translate to a large number of different subjects.

Thus, even though various systems and methods of identifying an immune response are known in the art, all or almost all of them suffer from various disadvantages. Consequently, there is a need to provide improved compositions and methods that provide improved systems and methods that help identify or even predict an immune response in an individual in a safe, simple, and expeditious manner.

### SUMMARY

The inventor has now discovered that an immune response can be ascertained or predicted in a subject, and preferably in a subject selected to receive or having received an antitumor vaccine, typically using whole blood of the subject and recombinant antigen as a starting material. Thus, contemplated compositions and methods are conceptually simple, significantly reduce risk and discomfort to the patient otherwise necessitated by a biopsy or surgery, and can provide results within a fraction of time (e.g., several days) otherwise required by in vivo observation (e.g., several months).

In one aspect, a method is disclosed of ascertaining an immune response against an antigen in a subject previously exposed to the antigen that includes a step of generating dendritic cells from peripheral blood of the subject, and exposing the dendritic cells to an antigen-containing composition to generate antigen presenting dendritic cells. In another step, T cells are isolated from peripheral blood of the subject, and the isolated T cells are then contacted with the antigen presenting dendritic cells. To expand antigen-reactive T cells, the isolated T cells and the antigen presenting dendritic cells are exposed to a cytokine-containing composition, and in still another step, the expanded antigen-reactive T cells are detected, whereby expanded antigen-reactive T cells ascertain an immune response against the antigen.

In some embodiments, the subject was previously exposed to a vaccine containing the antigen or nucleic acid encoding the antigen. For example, the vaccine containing the antigen may be a recombinant viral vaccine, a recombinant yeast vaccine, and/or a recombinant bacterial vaccine, and it is generally preferred (but not necessary) that the antigen is a patient and tumor specific neoantigen. Most typically, the dendritic cells are generated from monocytes in the peripheral blood.

In further embodiments, the antigen in the antigen-containing composition is a patient and tumor specific neoantigen, and/or the antigen in the antigen-containing composition is a full-length protein that contains a neoantigen. For example, the antigen-containing composition may be a recombinant antigen-containing composition. Where desired, the antigen-containing composition may comprise a polytope containing a plurality of distinct antigens or an antigen pool derived from a full-length protein. Most typically, the cytokine-containing composition may comprise IL7, IL15, and IL21, or the cytokine-containing composition may comprise an IL7/N803/IL21 TxM.

Among other suitable choices, the expanded antigen-reactive T cells may be detected using an ELISPOT assay or a FACS assay. As will also be readily appreciated, the expanded antigen-reactive T cells may be administered to the subject.

In another aspect, a method is disclosed of predicting a likely immune response against an antigen in a subject that is selected to receive a vaccine containing the antigen. Such method may include a step of generating dendritic cells from peripheral blood of the subject, and exposing the dendritic cells to an antigen-containing composition to generate antigen presenting dendritic cells, and a further step of isolating T cells from peripheral blood of the subject, and contacting the isolated T cells with the antigen presenting dendritic cells. The isolated T cells and the antigen presenting dendritic cells are then exposed to a cytokine-containing composition to expand antigen-reactive T cells, expanded antigen-reactive T cells are quantified, and in a still further step the subject is identified as a likely immune responder when the quantified expanded antigen-reactive T cells exceed a predetermined threshold quantity.

For example, the vaccine will typically be a recombinant viral vaccine, a recombinant yeast vaccine, and/or a recombinant bacterial vaccine, and the antigen will be a patient and tumor specific neoantigen. As noted above, the dendritic cells may be generated from monocytes in the peripheral blood. It is further contemplated that the antigen in the antigen-containing composition will be a patient and tumor specific neoantigen. Most typically, the antigen in the antigen-containing composition will be included in the vaccine. In still further contemplated embodiments, the vaccine may comprise a plurality of antigens and the antigen-containing composition comprises a plurality of antigens as an antigen pool or as a polytope, and the plurality of antigens in the vaccine is encoded or present as a polytope.

Preferably, but not necessarily, the cytokine-containing composition comprises IL7, IL15, and IL21, or the cytokine-containing composition comprises an IL7/N803/IL21 TxM. Likewise, it is preferred that the step of quantifying the expanded antigen-reactive T cells uses an ELISPOT assay or a FACS assay. Most typically, the predetermined threshold quantity is presence of the expanded antigen-reactive T cells at an abundance of at least 1.0% within an expansion culture.

Various objects, features, aspects, and advantages will become more apparent from the following detailed description of preferred embodiments, along with the accompanying drawing in which like numerals represent like components.

### BRIEF DESCRIPTION OF THE DRAWING

FIG. 1 schematically depicts a CMV viral particle and selected protein components.

FIG. 2 schematically depicts a CMV viral pp65 protein sequence, a peptide pool derived therefore, and a selected pp65₄₉₅₋₅₀₃ peptide fragment.

FIG. 3 schematically depicts an exemplary dextramer with dextran backbone that is labeled with fluorophores and further decorated with MEW proteins to which a peptide antigen is bound.

FIG. 4 depicts an exemplary expression strategy and synthetic nucleic acid that can be used to generate neoantigen (neoepitope) peptides in vitro.

FIG. 5 shows exemplary FACS results for antigen-reactive T cells and their enrichment after expansion.

FIGS. 6A and 6B are exemplary results for peptide titration for peripheral blood and using the peptide pool and an individual peptide fragment (6A) and interferon-γ production of antigen-reactive T cells in response to an individual peptide fragment and control (6B).

FIG. 7 shown an exemplary workflow and results for generation of expanded antigen-reactive T cells using monocyte derived dendritic cells and T cells from PBMC of a human subject.

FIG. 8 depicts exemplary results for detection of an antigen-reactive T cell fraction in expanded antigen-reactive T cells.

FIG. 9 depicts exemplary results for interferon-γ production of antigen-reactive T cells.

FIG. 10 depicts exemplary results that establish that antigen-reactive T cells recognize multiple epitopes from a polytope.

### DETAILED DESCRIPTION

The inventor has now discovered that successful generation of antigen-reactive T cells in a subject in response to immune therapy can be detected from whole blood in a conceptually simple and effective manner. More particularly, and based on the previously known antigens in the vaccine, an in vitro assay can be performed that uses monocyte derived dendritic cells of the subject that are exposed to the antigen or antigens (e.g., as an antigen pool or polytope), and the so generated antigen presenting dendritic cells are then contacted with T cells of the same subject to generate antigen-reactive T cells that are subsequently expanded using a specific cytokine-containing composition to obtain expanded antigen-reactive T cells for detection and/or quantification. As will be readily appreciated, presence of antigen-reactive T cells will be indicative of an immune response, and especially a therapeutically effective immune response where the quantity of expanded antigen-reactive T cells exceeds a predetermined threshold. Advantageously, detection and/or quantification can be performed using routine methods and equipment. Therefore, it should be recognized that the successful generation of antigen-reactive T cells in a subject in response to immune therapy can be verified (or even predicted) within only days from the subject receiving immune therapy. Consequently, and viewed from a different perspective, contemplated compositions and methods will significantly reduce the time spent between administration of a cancer vaccine and determination of its efficacy in a specific patient.

As will be readily appreciated, the nature of the immune therapy may vary considerably and will generally include direct or indirect administration of one or more disease related antigens. In preferred embodiments, the antigen is a cancer associated (e.g., MUC-1, CEA, etc.) or a cancer specific (e.g., PSA, PSMA, BRCA1, etc.) antigen, and most preferably a patient and tumor specific neoantigen. Thus, identification of suitable antigens may include a literature review, and more typically omics sequencing (e.g., whole genome sequencing, exon sequencing, RNA-seq, protein mass spectroscopy, etc.). In further preferred aspects, the neoantigens will be confirmed to be expressed in the tumor, and expressed neoantigens may be further filtered to those having a minimal binding affinity (e.g., equal or less than 500 nM, or equal or less than 200 nM, or equal or less than 100 nM) to the subjects HLA type. There are various manners of calculating minimal binding affinity and typical examples include NetMHC4.0, NetMHCpan, PSSNetMHCpan, MHCflurry, etc.

In still further contemplated aspects, suitable antigens in the cancer vaccine will be a plurality of antigens, typically arranged in a polytope in which neoantigens are sequentially arranged with interspersed (flexible) linker domains, typically having three to fifteen amino acids in length. For example, contemplated vaccine compositions especially include recombinant bacteria (e.g., E. coli, and especially E. coli engineered to lack LPS expression), viruses (e.g., Ad5, and especially Ad5[E1⁻Eb2⁻]), and/or yeast (e.g., Saccharomyces) that include a recombinant nucleic acid that encodes the antigen or polytope. Of course, it should be recognized that the subject may further receive additional therapeutic agents to stimulate an immune response such as immune stimulating cytokines (e.g., IL15, N803, etc.), checkpoint inhibitors (e.g., targeting CTLA4, PD-1, PD-L1, etc.), and cell based therapies such as T cells and/or NK cells (preferably genetically modified to express a chimeric antigen receptor or other tumor targeting entity).

Most typically, with respect to dendritic cells and T cells it is preferred that these cells are generated/obtained from peripheral blood. In most cases, PBMCs are obtained from the peripheral using standard methods well known in the art such as Ficoll density gradient centrifugation to obtain a buffy coat or leukapheresis. While dendritic cells may be isolated from PBMC, it is generally preferred that the dendritic cells are derived from monocytes in the PBMC (typically using anti-CD14 antibodies as is well known in the art) to so allow for relatively large quantities and relatively pure dendritic cell populations. Preparation of such monocyte derived dendritic cells is well known in the art (see e.g., J Vis Exp 2016) and will in most cases include a selected cytokine mixture including IL4 and GM-CSF. However, it should also be appreciated that the dendritic cells and/or T cells may also be from a heterologous source, and especially contemplated heterologous sources include HLA matched donors (e.g., with an HLA match to at least 4 digits or at least 6 digits for at least two HLA types (HLA-A, HLA-B, HLA-C, HLA-DR, HLA-DQ, HLA-DP)). In still further contemplated aspects, the dendritic cells and/or the T cells will be fresh cells, however, in some instances such cells may be previously frozen, particularly where the subject has a low count of dendritic cells and/or T cells due to chemotherapy.

It is still further contemplated that the blood draw may be performed prior to the patient receiving the immune therapy where prediction of an immune response is desired. On the other hand, where the patient has already received the anti-cancer vaccine, the blood draw may be performed between 1 and 7 days, or between 7 and 14 days, or between 14-28 days after first administration of the vaccine. Of course, it should be noted that more than a single blood draw and subsequent analysis is contemplated to allow for monitoring a dynamic immune response (e.g., where individual tests are used to monitor distinct neoantigens to identify antigen spread or to monitor strength of immune response over time to identify optimum response time and then switch to new and distinct vaccine).

Once blood is drawn and dendritic cells are generated, the dendritic cells can be contacted with the antigen or antigens in numerous manners. Among other options, the dendritic cells may be exposed to one or more individual purified antigens, to a at least partially purified polytope containing at least two antigens (typically separated by a linker peptide), or to crude extracts from cells expressing the antigen or polytope. In still further contemplated aspects, the antigen may also be prepared from an in vitro transcription/translation reaction, and so prepared antigens may be used directly in the transcription/translation mix or be further purified. Upon suitable exposure time, typically between 2-6 hours, or between 6-12 hours, or between 12-24 hours (and in some cases even longer), T cells will be added to the pulsed dendritic cells. Most typically, the T cells will be present relative to the dendritic cells at a ratio of about 10:1, or 7:1, or 5:1, or 3:1, or 1:1, or 1:3, or 1:5, or 1:7, or 1:10. Where desired, the exposure of the T cells to the primed dendritic cells may further include one or more immune stimulating cytokines.

Regardless of the specific exposure, so activated T cells are then expanded (after optional isolation using a Ficoll gradient) in an expansion medium that contains a cytokine composition to preferentially stimulate cell division of activated T cells. Most typically, the cytokine composition will comprise IL7, IL15, and IL21, or an IL7/N803/IL21 TxM. Expansion will be performed over a period of about 7-20 days, typically for less than two weeks with media change every 2-5 days (e.g., 3-4 days).

Upon conclusion of expansion of the antigen reactive T cells, the population of antigen-reactive T cells can then be determined using various methods well known in the art. However, it is generally preferred that the determination will use an ELISPOT assay and/or a FACS assay in which a labeled construct comprising MHC-bound neoantigen is used as a fluorescence marker as is described in more detail below. As will be readily appreciated, such methods not only provide a qualitative result, but may also be used to quantify the immune response in a subject. Most typically, a threshold value is established that is reflective or predictive of an immune response (e.g., expanded antigen-reactive T cells present at an abundance of at least 0.5%, or at least 1.0%, or at least 1.5%, or at least 3% within an expansion culture).

### Examples

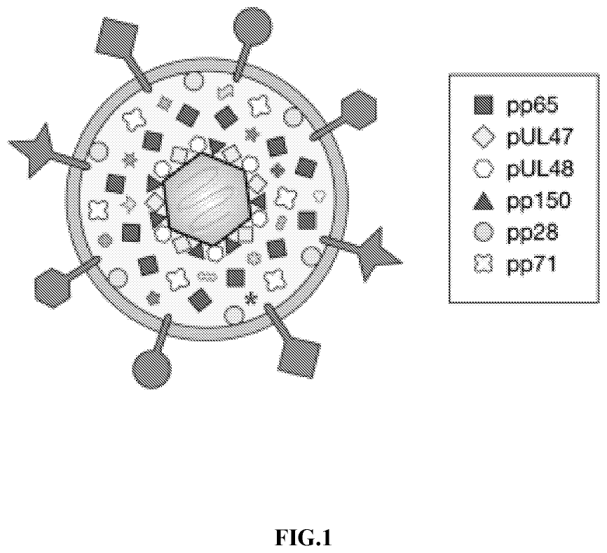

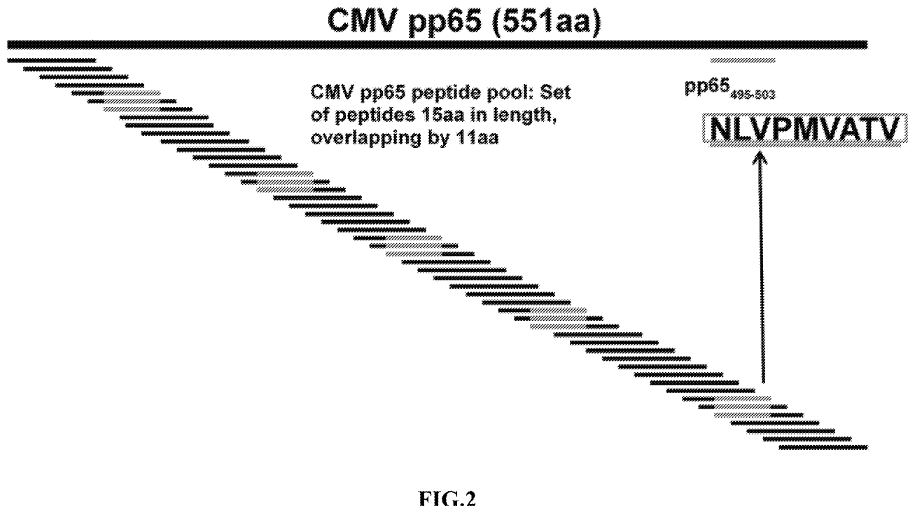

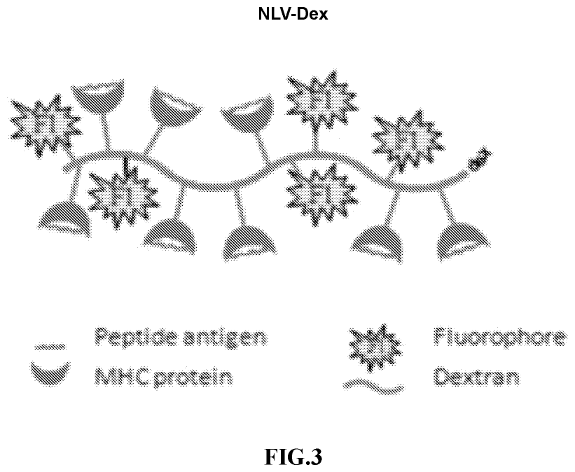

The following examples use CMV as a model system for a viral vaccine in human, which is a common and well characterized virus. In particular, the 65 kDa lower matrix phosphoprotein (pp65) is the main component of the enveloped subviral particle and an immunodominant antigen recognized by both CD4 and CD8 T cells as is schematically shown in FIG. 1. For the studies presented below, the full-length sequence was used, a peptide pool of overlapping sequences spanning the entire sequence (e.g., 15 amino acids in length, overlapping by 11 amino acids), as well as a selected peptide (e.g., pp65₄₉₅₋₅₀₃), which was in vitro transcribed and translated) as is exemplarily shown in FIG. 2. For cells, peripheral blood from CMV-seropositive, HLA A2 0201 positive subjects was used unless specified otherwise. Cell detection and quantification was performed using an ELISPOT assay and FACS with activated T cell staining using a MHC (HLA A2 0201) decorated dextramer to which the pp65₄₉₅₋₅₀₃ peptide was bound as is schematically shown in FIG. 3.

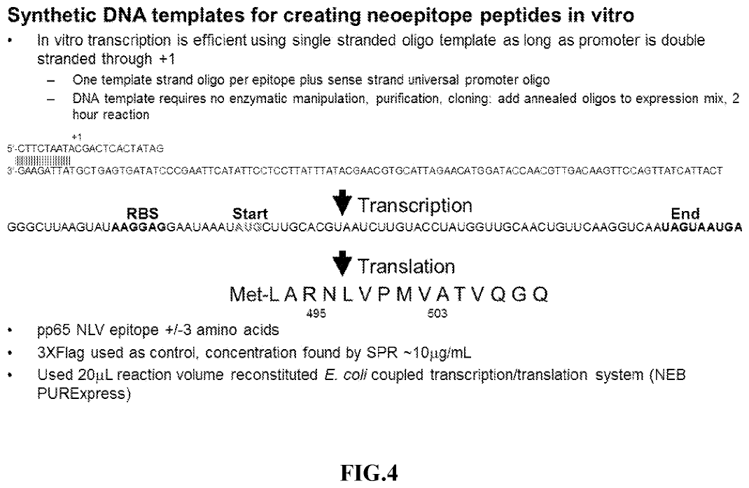

While synthetic peptides can be used for all of the single peptide, the examples below employed a recombinant pp65₄₉₅₋₅₀₃ peptide that was produced from a construct as is shown in FIG. 4. Here, a synthetic nucleic acid was prepared and hybridized to a universal promoter primer to generate the sense strand RNA that was translated in vitro to the corresponding peptide (here with 3 additional amino acids at the N- and C-terminus). Unless otherwise noted, generation of dendritic cells from peripheral blood monocytes, isolation/enrichment of peripheral blood monocytes and T cells, cell culture, ELISPOT, and FACS analysis followed standard methods known in the art. Expansion of antigen reactive cells used an IL7/N803/IL21 TxM (as described in US2019/0300591, incorporated by reference herein).

More particularly, peripheral blood was drawn from two CMV-seropositive, HLA A2 0201 positive subjects using venipuncture. Monocytes were isolated using the EasySep™ Human Monocyte Isolation Kit (commercially available from Stem cell Technologies) or following other known methods of CD14-based enrichment from PBMCs. To further mature and differentiate the monocytes to dendritic cells, IL-4, GM-CSF, and TNF-α were employed. To that end, monocytes were treated with the CellXVivo Human Monocyte-derived DC Differentiation Kit (commercially available from RD Systems).

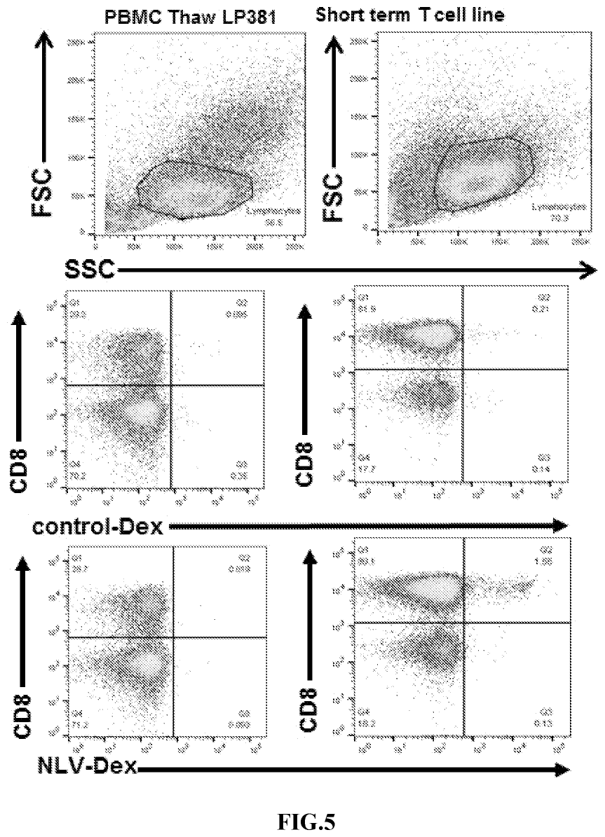

The so prepared dendritic cells were then exposed to pp65 as full-length protein (see full length sequence below), as polytope (see full length sequence below), as crude cell lysate of recombinant E. coli expressing pp65, as His-purified pp65, as pp65 peptide pool as described above, or as pp65₄₉₅₋₅₀₃ peptide fragment (see FIG. 3). Specifically, and with regard to FIG. 5, the frequency of pp65₄₉₅₋₅₀₃ reactive T cells in PBMC (left column) and a T cell line (right column) generated from the same HLA-A2, CMV-seropositive subject was measured by flow cytometry. Here, the cells were labeled with a dextramer (Immudex.com), specific for pp65₄₉₅₋₅₀₃ peptide presented by human HLA-A2, together with anti-CD8 antibody. Control dextramer (control-dex) was the dextramer without peptide. The top panels in FIG. 5 show the scatter (forward scatter-FSC; side scatter-SSC) properties of the cells, and the middle panels show the labeling with control dextramer. The lower panels show the labeling with the pp65₄₉₅₋₅₀₃ dextramer (NLV-dex).

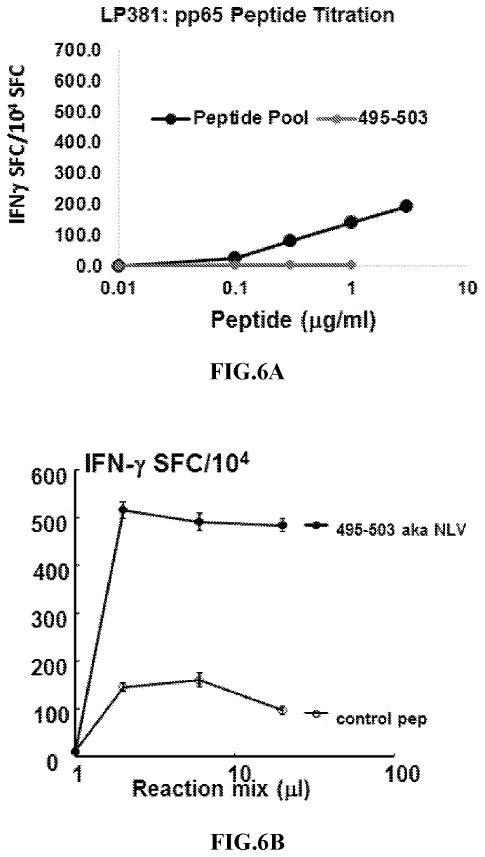

FIGS. 6A and 6B depicts the frequency of pp65₄₉₅₋₅₀₃ reactive T cells in PBMC and a T cell line generated from the same HLA-A2, CMV-seropositive subject measured by ELISPOT. FIG. 6A shows results for PBMC from a HLA-A2, CMV-seropositive subject were pulsed with a pool of overlapping peptides (11 amino acids in length) spanning the sequence of CMV pp65 (peptide pool) or the pp65₄₉₅₋₅₀₃ peptide (495-503). Cells secreting IFN-γ were detected using ELISPOT. FIG. 6B depicts results for a T cell line generated using the pool of overlapping peptides (11 amino acids in length) spanning the sequence of CMV pp65 was evaluated for the frequency of pp65₄₉₅₋₅₀₃ peptide-reactive T cells. Antigen presenting cells (autologous monocyte-derived dendritic cells) were pulsed with antigen (in vitro transcribed and translated peptide 495-503, aka NLV) or control antigen (in vitro transcribed and translated control peptide). Pulsed APC were then cultured with a T cell line and the frequency of antigen-reactive T cells was detected by IFN-γ secretion using ELISPOT.

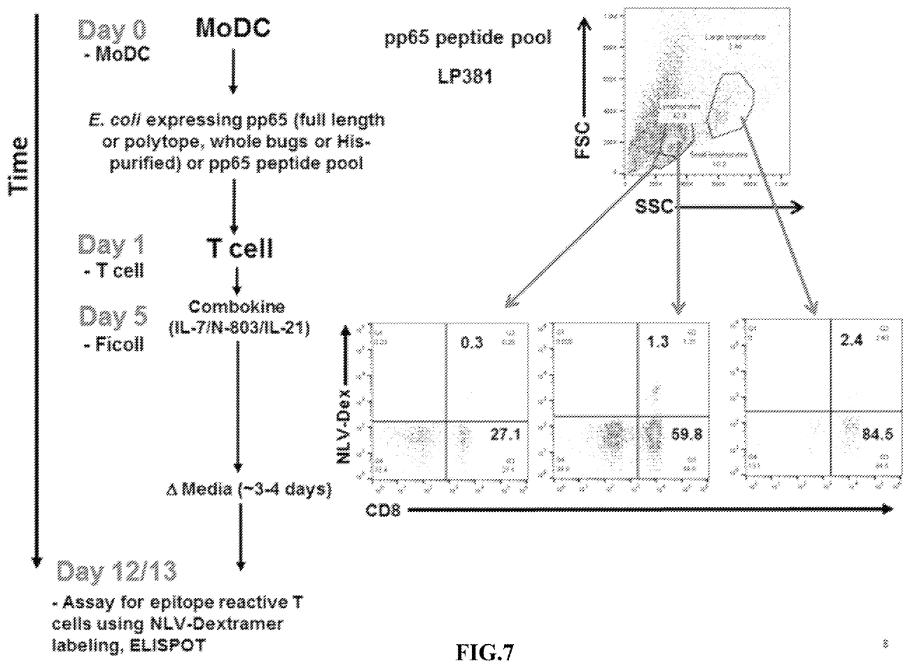

As can be readily seen from FIG. 5, antigen-reactive T cells are present at a relatively low frequency in peripheral blood, but can be enriched by generating in vitro short term T cell lines. More particularly, as is shown in FIG. 7, monocyte derived dendritic cells will activate T cells upon contact with the antigen (here: pp65 as full-length protein (see full length sequence below), as polytope (see full length sequence below), as crude cell lysate of recombinant E. coli expressing pp65, as His-purified pp65, as pp65 peptide pool as described above, or as pp65₄₉₅₋₅₀₃ peptide fragment. More specifically, dendritic cells were pulsed (16 hr) with antigen in culture medium (RPMI containing 10% fetal bovine serum) at concentrations previously determined to stimulate T cells. Pulsed dendritic cells were washed, counted, then added in a 1:5 ratio of dendritic cells to T cells. Note that the term “combokine” used in the figure is refers to a mixture of separate cytokines, N-803, IL-21. Interestingly, when a TxM (with IL-7/15/21 portions as described in US2019/0300591, incorporated by reference herein) was used, substantially the same results were obtained as compared to the combination of IL-7, N-803, IL-21.

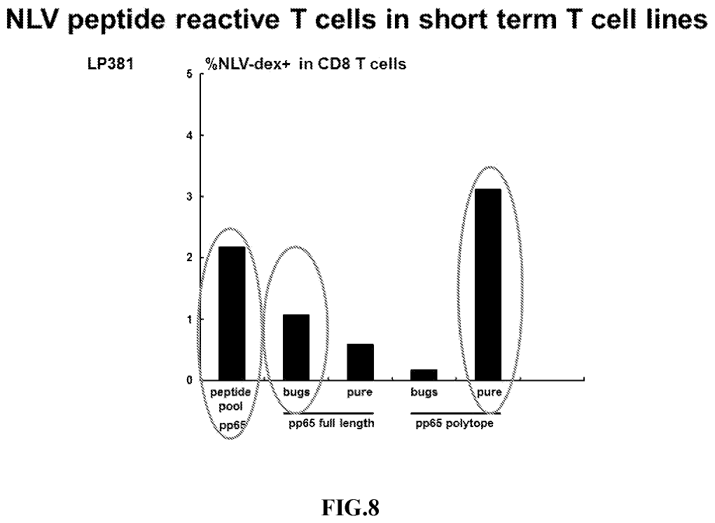

Antigen reactive T cells were then detected and/or quantified using a standard ELISPOT assay as well as a FACS analysis using fluorescence labeled dextramer that was decorated with MHC to which was bound the peptide antigen (e.g., pp65₄₉₅₋₅₀₃ peptide fragment). As can be seen from the FACS results shown in FIG. 7, antigen reactive T cells could be detected and quantified after expansion of activated T cells. FIG. 8 depicts further exemplary results for expanded antigen reactive T cells (short term T cell lines) where the dextramer of FIG. 3 was used to detect and quantify a proportion of antigen reactive cells. Here, the examples illustrate results for dendritic cells incubated with the peptide pool (see FIG. 2), the full length pp65 protein (either as crude extract or as isolated protein), and the recombinant polytope (see polytope sequence below) as crude extract or as isolated polytope. As is readily evident, the peptide pool and isolated polytope were effective in eliciting a significant immune response, which mirrors a recombinant polytope vaccine.

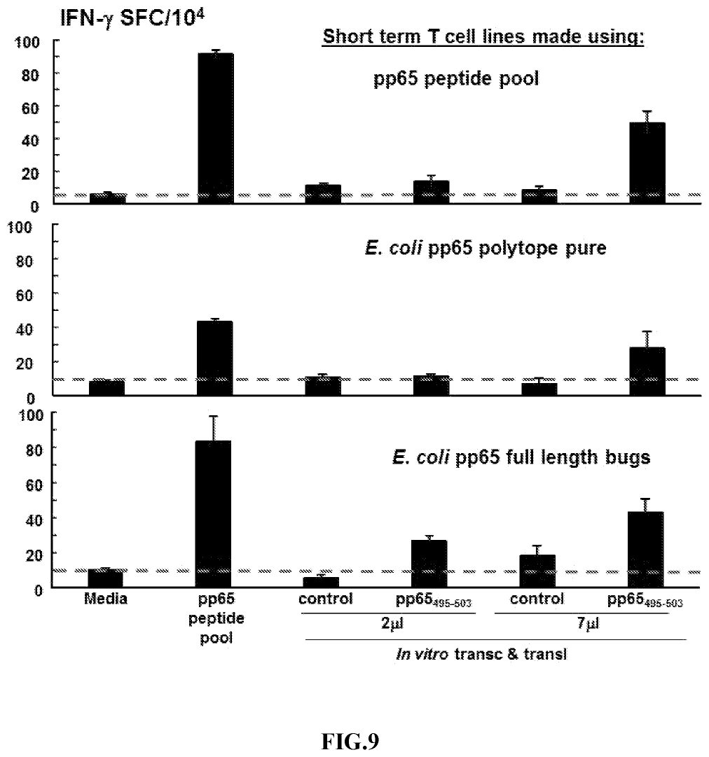

In particular, T cell lines were generated as described for FIG. 7 above. The frequency of pp65₄₉₅₋₅₀₃-reactive T cells was determined as in FIG. 5 and FIG. 7 using the NLV-dextramer and flow cytometry. The cell lines circled in ovals were selected for their relatively higher frequency of pp65₄₉₅₋₅₀₃-reactive T cells so that they might be used to evaluate peptide reactivity when peptide is provided through in vitro transcription and translation as shown in FIG. 9. Here, FIG. 9 depicts exemplary results for interferon-γ, once more demonstrating that the peptide pool and isolated polytope were effective in eliciting a significant immune response. More particularly, the frequency of pp65₄₉₅₋₅₀₃-reactive T cells was determined using ELISPOT. Autologous monocyte derived dendritic cells were pulsed with antigens (as indicated below the bar graphs), then T cells were added (1:5 ratio of dendritic cells to T cells) and IFN-γ secretion measured. The dotted line indicates the frequency of IFN-γ spot forming cells (SFC) when no antigen is added, i.e. media added only.

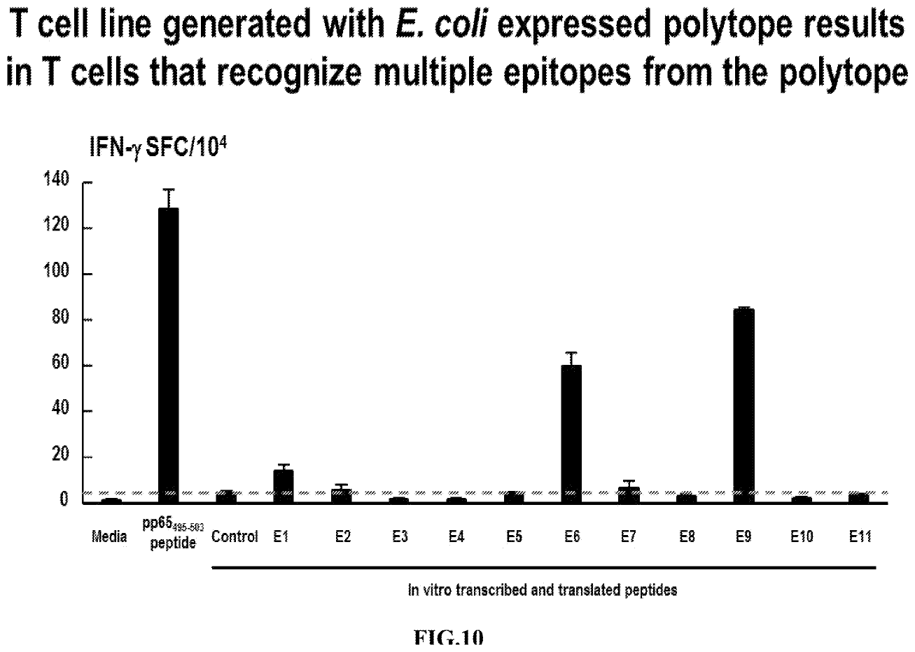

In addition, as can be seen from the key data shown in FIG. 10, that T cells generated with E. coli expressed polytope (same T cells as those generated in FIG. 9, middle panel) result in T cells that recognize multiple epitopes from the polytope. With further reference to FIG. 10, it should be appreciated that that epitopes E1, E6, and E9 were recognized by T cells in the T cell line. FIG. 11 shows the amino acid sequences for the peptides translated for the ELISPOT tested in FIG. 10. Such results strongly indicate that T cells generated with a polytope of T cell epitopes expressed in E. coli (LPS-deficient) are actually composed of T cells with multiple specificities. This type of result is expected to be replicated with patient neoepitopes and patient blood.

Sequences

The amino acid sequence of the pp65 full length protein is shown in SEQ ID NO:1.

The amino acid sequence of the pp65 Polytope (31mers with flexible linker) is shown in SEQ ID NO:2. The calculated binding affinities for sequences within the polytope are shown below:

TABLE 1

| | Predicted binding |
|---|---|
| sequence | affinity (HLA-A2 0201) |
| NLVPMVATV (SEQ ID NO: 3) | 29 |
| YTSAFVFPT (SEQ ID NO: 4) | 33 |
| RIFAELEGV (SEQ ID NO: 5) | 34 |
| LMNGQQIFL (SEQ ID NO: 6) | 44 |
| MLNIPSINV (SEQ ID NO: 7) | 54 |
| QMWQARLTV (SEQ ID NO: 8) | 64 |
| RLLQTGIHV (SEQ ID NO: 9) | 66 |
| SIYVYALPL (SEQID NO: 10) | 79 |
| ALFFFDIDL (SEQID NO: 11) | 82 |
| IMLDVAFTS (SEQ ID NO: 12) | 85 |
| YLESFCEDV (SEQ ID NO: 13) | 133 |

TABLE 2

| | Predicted |
|---|---|
| | binding |
| | affinity |
| Sequence | (HLA-DRB10101) |
| TGIHVRVSQPSLILVSQ (SEQ ID NO: 14) | 5.6 |
| SHEHFGLLCPKSIPGL (SEQ ID NO: 15) | 5.6 |
| ERNGFTVLCPKNMIIK (SEQ ID NO: 16) | 7.5 |
| YALPLKMLNIPSINVHH (SEQ ID NO: 17) | 7.6 |

An exemplary synthetic DNA template for creating neoepitope peptides in vitro is depicted below, and the corresponding sequences are shown in SEQ ID NO:18, SEQ ID NO:19, SEQ ID NO:20, and SEQ ID NO:21 respectively. Table 3 lists a variety of other sequence constructs used in the experiment of FIG. 10.

TABLE 3
Table 3. IVT&T peptides: Met-25 mer-GKCCPGCC

| | | |
|---|---|---|
| pp65-1 | ETRLLQTGIHVRVSQPSLILVSQYT | (SEQ ID NO: 22) |
| pp65-2 | QEPMSIYVYALPLKMLNIPSINVHH | (SEQ ID NO: 23) |
| pp65-3 | AVIHASGKQMWQARLTVSGLAWTRQ | (SEQ ID NO: 24) |
| pp65-4 | QWKEPDVYYTSAFVFPTKDVALRHV | (SEQ ID NO: 25) |
| pp65-5 | IGDQYVKVYLESFCEDVPSGKLFMH | (SEQ ID NO: 26) |
| pp65-6 | FMRPHERNGFTVLCPKNMIIKPGKI | (SEQ ID NO: 27) |
| pp65-7 | IMLDVAFTSHEHFGLLCPKSIPGLS | (SEQ ID NO: 28) |
| pp65-8 | LRQYDPVAALFFFDIDLLLQRGPQY | (SEQ ID NO: 29) |
| pp65-9 | WQAGILARNLVPMVATVQGQNLKYQ | (SEQ ID NO: 30) |
| pp65-10 | FWDANDIYRIFAELEGVWQPAAQPK | (SEQ ID NO: 31) |
| pp65-11 | GLSISGNLLMNGQQIFLEVQAIRET | (SEQ ID NO: 32) |

As shown in FIG. 4, in-vitro transcription is efficient using single stranded oligo template as long as promoter is double stranded through +1. One template strand oligo per epitope plus sense strand universal promoter oligo was used. DNA template requires no enzymatic manipulation, purification, cloning. Rather the reaction required simply adding annealed oligos to expression mix, and the mixture was allowed to react for 2 hours. In the exemplary method for creating neoepitope peptides in vitro as shown in FIG. 3, pp65 NLV epitope +/−3 amino acids were used, 3×Flag used as control, concentration found by SPR˜10 mg/mL, and 20 mL reaction volume was used to reconstitute E. coli coupled transcription/translation system (NEB PURExpress)

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the full scope of the present disclosure, and does not pose a limitation on the scope of the invention otherwise claimed. No language in the specification should be construed as indicating any non-claimed element essential to the practice of the claimed invention.

It should be apparent to those skilled in the art that many more modifications besides those already described are possible without departing from the full scope of the concepts disclosed herein. The disclosed subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1. A method of ascertaining an immune response against an antigen in a subject previously exposed to the antigen, comprising:
generating dendritic cells from peripheral blood of the subject, and exposing the dendritic cells to an antigen-containing composition to generate antigen presenting dendritic cells;
isolating T cells from peripheral blood of the subject, and contacting the isolated T cells with the antigen presenting dendritic cells;
exposing the isolated T cells and the antigen presenting dendritic cells to a cytokine-containing composition to expand antigen-reactive T cells; and
detecting the expanded antigen-reactive T cells.

2. The method of claim 1 wherein the subject previously exposed to the antigen is a subject that was previously exposed to a vaccine containing the antigen.

3. The method of claim 2 wherein the vaccine containing the antigen is a recombinant viral vaccine, a recombinant yeast vaccine, and/or a recombinant bacterial vaccine, and wherein the antigen is a patient and tumor specific neoantigen.

4. The method of claim 1, wherein the dendritic cells are generated from monocytes in the peripheral blood.

5. The method of claim 1, wherein the antigen in the antigen-containing composition is a patient and tumor specific neoantigen.

6. The method of claim 1, wherein the antigen in the antigen-containing composition is a full-length protein that contains a neoantigen.

7. The method of claim 1, wherein the antigen-containing composition is a recombinant antigen-containing composition.

8. The method of claim 1, wherein the antigen-containing composition comprises a polytope containing a plurality of distinct antigens or an antigen pool derived from a full-length protein.

9. The method of claim 1, wherein the cytokine-containing composition comprises IL7, IL15, and IL21, or wherein the cytokine-containing composition comprises an IL7/N803/IL21 TxM.

10. The method of claim 1, wherein detecting the expanded antigen-reactive T cells comprises an ELISPOT assay or a FACS assay.

11. The method of claim 1, further comprising a step of administering the expanded antigen-reactive T cells to the subject.

12. A method of predicting a likely immune response against an antigen in a subject selected to receive a vaccine containing the antigen, comprising:
generating dendritic cells from peripheral blood of the subject, and exposing the dendritic cells to an antigen-containing composition to generate antigen presenting dendritic cells;
isolating T cells from peripheral blood of the subject, and contacting the isolated T cells with the antigen presenting dendritic cells;
exposing the isolated T cells and the antigen presenting dendritic cells to a cytokine-containing composition to expand antigen-reactive T cells;
quantifying the expanded antigen-reactive T cells; and
identifying the subject as a likely immune responder when the quantified expanded antigen-reactive T cells exceed a predetermined threshold quantity.

13. The method of claim 12 wherein the vaccine containing the antigen is a recombinant viral vaccine, a recombinant yeast vaccine, and/or a recombinant bacterial vaccine, and wherein the antigen is a patient and tumor specific neoantigen.

14. The method of claim 12, wherein the dendritic cells are generated from monocytes in the peripheral blood.

15. The method of claim 12, wherein the antigen in the antigen-containing composition is a patient and tumor specific neoantigen.

16. The method of claim 12, wherein the antigen in the antigen-containing composition is included in the vaccine.

17. The method of claim 12, wherein the vaccine comprises a plurality of antigens and wherein the antigen-containing composition comprises a plurality of antigens as an antigen pool or as a polytope, and wherein the plurality of antigens in the vaccine are encoded or present as a polytope.

18. The method of claim 1, wherein the cytokine-containing composition comprises IL7, IL15, and IL21, or wherein the cytokine-containing composition comprises an IL7/N803/IL21 TxM.

19. The method of claim 1, wherein quantifying the expanded antigen-reactive T cells comprises an ELISPOT assay or a FACS assay.

20. The method of claim 1, wherein the predetermined threshold quantity is presence of the expanded antigen-reactive T cells at an abundance of at least 1.0% within an expansion culture.


SEQUENCE LISTING (32 sequences)

SEQ ID NO: 1 - protein, 624 aa, Artificial Sequence
  pp65 full length protein
MASVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRKRKSASSATACTAGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRGAAAAEVSGLEQLESIINFEKLTEWTSSNVAAAAMTEQQWNFAGIEAAASAIQGNVTSIHSLLDAAAAHHHHHH

SEQ ID NO: 2 - protein, 397 aa, Artificial Sequence
  pp65 Polytope (31mers with flexible linker)
MLPHETRLLQTGIHVRVSQPSLILVSQYTPDSGPGPGCPSQEPMSIYVYALPLKMLNIPSINVHHYPSGPGPGVADAVIHASGKQMWQARLTVSGLAWTRQQNQGPGPGQQNQWKEPDVYYTSAFVFPTKDVALRHVVCAGPGPGQVIGDQYVKVYLESFCEDVPSGKLFMHVTLGPGPGTRNPQPFMRPHERNGFTVLCPKNMIIKPGKSGPGPGISHIMLDVAFTSHEHFGLLCPKSIPGLSISGGPGPGTVELRQYDPVAALFFFDIDLLLQRGPQYSEHGPGPGWPPWQAGILARNLVPMVATVQGQNLKYQEFFGPGPGQEFFWDANDIYRIFAELEGVWQPAAQPKRRRGPGPGSIPGLSISGNLLMNGQQIFLEVQAIRETVELHHHHHH

SEQ ID NO: 3 - protein, 9 aa, Artificial Sequence
  PP65 peptide
NLVPMVATV

SEQ ID NO: 4 - protein, 9 aa, Artificial Sequence
  PP65 peptide2
YTSAFVFPT

SEQ ID NO: 5 - protein, 9 aa, Artificial Sequence
  PP65 peptide3
RIFAELEGV

SEQ ID NO: 6 - protein, 9 aa, Artificial Sequence
  PP65 peptide4
LMNGQQIFL

SEQ ID NO: 7 - protein, 9 aa, Artificial Sequence
  PP65 peptide5
MLNIPSINV

SEQ ID NO: 8 - protein, 9 aa, Artificial Sequence
  PP65 peptide6
QMWQARLTV

SEQ ID NO: 9 - protein, 9 aa, Artificial Sequence
  PP65 peptide7
RLLQTGIHV

SEQ ID NO: 10 - protein, 9 aa, Artificial Sequence
  PP65 peptide8
SIYVYALPL

SEQ ID NO: 11 - protein, 9 aa, Artificial Sequence
  PP65 peptide9
ALFFFDIDL

SEQ ID NO: 12 - protein, 9 aa, Artificial Sequence
  PP65 peptide10
IMLDVAFTS

SEQ ID NO: 13 - protein, 9 aa, Artificial Sequence
  PP65 peptide11
YLESFCEDV

SEQ ID NO: 14 - protein, 17 aa, Artificial Sequence
  PP65 peptide12
TGIHVRVSQPSLILVSQ

SEQ ID NO: 15 - protein, 16 aa, Artificial Sequence
  PP65 peptide13
SHEHFGLLCPKSIPGL

SEQ ID NO: 16 - protein, 16 aa, Artificial Sequence
  PP65 peptide14
ERNGFTVLCPKNMIIK

SEQ ID NO: 17 - protein, 17 aa, Artificial Sequence
  PP65 peptide15
YALPLKMLNIPSINVHH

SEQ ID NO: 18 - DNA, 22 bp, Artificial Sequence
  transcription seq1
cttctaatacgactcactatag

SEQ ID NO: 19 - DNA, 104 bp, Artificial Sequence
  transcription seq2
gaagattatgctgagtgatatcccgaattcatattcctccttatttatacgaacgtgcattagaacatggataccaacgttgacaagttccagttatcattact

SEQ ID NO: 20 - RNA, 83 nt, Artificial Sequence
  transcription seq3
gggcuuaaguauaaggaggaauaaauaugcuugcacguaaucuuguaccuaugguugcaacuguucaaggucaauaguaauga

SEQ ID NO: 21 - protein, 16 aa, Artificial Sequence
  transcription seq4
MLARNLVPMVATVQGQ

SEQ ID NO: 22 - protein, 25 aa, Artificial Sequence
  IVT&T peptide1
ETRLLQTGIHVRVSQPSLILVSQYT

SEQ ID NO: 23 - protein, 25 aa, Artificial Sequence
  IVT&T peptide2
QEPMSIYVYALPLKMLNIPSINVHH

SEQ ID NO: 24 - protein, 25 aa, Artificial Sequence
  IVT&T peptide3
AVIHASGKQMWQARLTVSGLAWTRQ

SEQ ID NO: 25 - protein, 25 aa, Artificial Sequence
  IVT&T peptide4
QWKEPDVYYTSAFVFPTKDVALRHV

SEQ ID NO: 26 - protein, 25 aa, Artificial Sequence
  IVT&T peptide5
IGDQYVKVYLESFCEDVPSGKLFMH

SEQ ID NO: 27 - protein, 25 aa, Artificial Sequence
  IVT&T peptide6
FMRPHERNGFTVLCPKNMIIKPGKI

SEQ ID NO: 28 - protein, 25 aa, Artificial Sequence
  IVT&T peptide7
IMLDVAFTSHEHFGLLCPKSIPGLS

SEQ ID NO: 29 - protein, 25 aa, Artificial Sequence
  IVT&T peptide8
LRQYDPVAALFFFDIDLLLQRGPQY

SEQ ID NO: 30 - protein, 25 aa, Artificial Sequence
  IVT&T peptide9
WQAGILARNLVPMVATVQGQNLKYQ

SEQ ID NO: 31 - protein, 25 aa, Artificial Sequence
  IVT&T peptide10
FWDANDIYRIFAELEGVWQPAAQPK

SEQ ID NO: 32 - protein, 25 aa, Artificial Sequence
  IVT&T peptide11
GLSISGNLLMNGQQIFLEVQAIRET